(19) United States
(12) Patent Application Publication
(10) Pub. No.: US 2002/0131957 A1
(43) Pub. Date: Sep. 19, 2002

(54) Cryopreservation of sperm

(76) Inventors: William Gavin, Dudley, MA (US); Stephen Blash, North Oxford, MA (US); Christina A. Cammuso, Holden, MA (US); David Melican, Fiskdale, MA (US)

Correspondence Address: LOUIS MYERS, Fish & Richardson P.C., 225 Franklin Street, Boston, MA, 02110-2804, US

(21) Appl. No.: 09/927,824

(22) Filed: Aug. 10, 2001

Related U.S. Application Data
(60) Provisional application No. 60/224,393, filed on Aug. 10, 2000.

(51) Int. Cl. A01N 1/02; A61K 45/00

(52) U.S. Cl. 424/93.7; 435/2

(57) ABSTRACT

The invention features methods of cryopreserving sperm. The methods include providing a sample of sperm; cooling the sperm to a first temperature; maintaining the sperm sample at the first temperature; cooling the sperm to a second temperature; maintaining the sperm at the second temperature; and storing the sperm at a third temperature. These methods can be used to cryopreserve sperm from mammals, e.g., transgenic mammals and can be to preserve sperm for subsequent artificial insemination or in vitro fertilization.

### RELATED APPLICATIONS

This application claims priority to U.S. provisional application number No. 60/224,393 filed on Aug. 10, 2000 the contents of which are incorporated herein by reference.

### BACKGROUND OF THE INVENTION

The ability to modify animal genomes through transgenic technology has opened new avenues for medical applications. By targeting the expression of biomedical proteins to the mammary gland of farm animals, low-cost production of high quantities of valuable therapeutic proteins is now possible (Houdebine (1995) Reprod. Nutr. Dev. 35:609-617; Maga et al. (1995) Bio/Technology, 13:1452-1457; Echelard (1996) Curr.Op.Biotechnol. 7:536-540; Young et al. (1997) BioPharm. 10:34-38). Although the total sales for the top fifteen biopharmaceuticals in 1996 were $7.5 billion, expectations are that this number will continue to rise in the future. Med. Ad News 16:30. Transgenic technology is applicable and attractive for proteins that, whether due to high unit dosage requirements, frequency of administration, or large patient populations, are needed in high volume, and also to complex proteins that are difficult to produce in commercially viable quantities using traditional cell culture methods. In addition, the production of human pharmaceuticals in the milk of transgenic farm animals solves many of the problems associated with microbial bioreactors, e.g., lack of post-translational modifications, improper folding, high purification costs, or animal cell bioreactors, e.g., high capital costs, expensive culture media, low yields.

The production of founder transgenic animals, however, can be expensive. Male animals with valuable genetics are often lost unexpectedly. These unexpected deaths can present the owner with a great financial loss, and more importantly the loss of the animal's genetics if offspring were not produced or semen cryopreserved. In a transgenic production setting, the loss of a founder male has a significant economic impact and disrupts the time frame for projects.

The genetic material from many species has been preserved and passed on by using artificial insemination and in vitro fertilization techniques. The process of freezing spermatozoa can be harsh as a result of thermal, osmotic, and/or mechanical shock to the cell, and the formation of crystals, which can damage cellular structures, particularly the plasma membrane. In addition, the process of freezing and thawing causes dehydration of the cell with potential for cellular damage. Methods that overcome these obstacles are useful for preserving sperm for any number of purposes, e.g., medical, commercial, and agricultural purposes.

### SUMMARY OF THE INVENTION

The invention is based, in part, on the discovery that viable sperm can be preserved by cooling a sperm sample to a first temperature at a rate sufficiently slow that the metabolic rate of sperm is decreased, and then freezing the sperm sample at a second temperature prior to storing the sample, e.g., in liquid nitrogen. The low temperature preservation of such gametes allows their utilization at a later time. It was also found that by cooling the sperm to the first temperature prior to addition of the glycerol, the sperm are protected from glycerol toxicity. Such an invention has broad applications in the areas of agriculture, pharmaceuticals, natural resource conservation, and veterinary and human medicine. In particular, the method facilitates the preservation of individual genetic compositions.

Accordingly, in one aspect, the invention features a method of providing sperm. The method includes cooling a sample, which includes sperm, to a first temperature sufficient to protect sperm from glycerol addition and at a rate sufficiently slow to decrease the metabolic rate of sperm to thereby provide a cooled sperm sample. The method further includes adding a solution which includes glycerol, and freezing the cooled sperm sample to a second temperature for a sufficient period of time to equilibrate glycerol and sperm to thereby provide a frozen sperm sample, such that the sperm is preserved.

In one embodiment, the method includes providing a sample of semen, e.g., semen obtained from live animals. In another embodiment, the sperm sample is obtained by extraction from the epididymis, e.g., at necropsy. The method can further include isolating the sperm from the provided sample, e.g., by centrifugation. In one embodiment, the sperm sample is at a temperature of between about 27° C. and about 38° C., preferably about 37° C., prior to cooling. The sperm sample can be obtained from a mammal, e.g., a goat, a cow, a sheep, a rabbit, a pig, or a mouse, preferably a goat or rabbit. In a preferred embodiment, the mammal is a transgenic mammal, e.g., a mammal containing a transgene encoding a polypeptide. The polypeptide can be any protein whose expression is desired in a transgenic mammal, including any of: α-1 proteinase inhibitor, alkaline phosphatase, angiogenin, antibodies, extracellular superoxide dismutase, fibrinogen, glucocerebrosidase, glutamate decarboxylase, human serum albumin, myelin basic protein, proinsulin, soluble CD4, lactoferrin, lactoglobulin, lysozyme, lactalbumin, erythrpoietin, tissue plasminogen activator, human growth factor, antithrombin III, insulin, prolactin, and α1-antitrypsin. The transgene can further include a promoter, e.g., a milk specific promoter. The milk specific promoter can be any of: a casein, a whey acid protein, an α-lactalbumin, a β-lactoglobin, or a lactoferrin promoter.

In one embodiment, the method includes providing the sample to be cooled in a cryoprotectant buffer. In a preferred embodiment, the cryoprotectant buffer lacks glycerol. The cryoprotectant buffer can include egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In one embodiment, the first temperature can be between about 0° C. to about 10° C., preferably about 1° C. to about 8° C., more preferably about 5° C. In a preferred embodiment, the sperm sample is cooled to the first temperature at a rate of about 0.2° C. to about 0.5° C. per minute, preferably about 0.5° C. per minute. In another embodiment, the sperm sample is cooled over the course of about 1.5 hours to about 4 hours, preferably about 1.5 hours. In another embodiment, the sperm sample is maintained at the first temperature for a period of time, e.g., between about 4 hours and about 21 hours, preferably about 4 hours.

In one embodiment, the solution containing glycerol has a concentration of glycerol of about 5% to 10% glycerol, preferably 7% glycerol. The solution can be the cryoprotectant buffer used prior to the cooling step which further includes glycerol. The cryoprotectant buffer can include egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In a preferred embodiment, the second temperature can be about −40° C. to about −100° C., e.g., about −60° C. to about −90° C., preferably about −80° C. In another embodiment, the frozen sperm sample is maintained at the second temperature for about 7 minutes to 20 minutes, preferably for about 10 minutes to about 18 minutes, more preferably for about 15 minutes.

In one embodiment, the method further comprises placing the frozen sperm sample at a third temperature of about −180° C. to about −200° C., e.g., about −196° C., e.g., in liquid nitrogen. The sperm sample can be maintained at the third temperature until further use. In another embodiment, the frozen sperm sample is thawed from the third temperature. Preferably, the sample is thawed at about 27° C. to about 38° C., for about 1 minute to about 5 minutes, preferably for about 1.5 minutes. In one embodiment, the percentage of viable sperm after thawing is about 20%, 30%, 40%, 50%, 55%, 60%, 65%, 70%, 75%, 80%, 85%, 90%, 95%, 98%, 99%, or 100%.

Another aspect of the invention features a method for preserving sperm. The method includes: cooling a sperm sample to a first temperature of between about 2° C. to about 10° C. at a rate sufficiently slow to decrease the metabolic rate of sperm to produce cooled sperm; freezing the cooled sperm at a second temperature of between about −60° C. to about −90° C.; and storing the frozen sperm at a temperature of between about −180° C. to about −220° C., preferably −196° C.

In one embodiment, the method includes providing a sample of semen, e.g., semen obtained from live animals. In another embodiment, the sperm sample is obtained by extraction from the epididymis, e.g., at necropsy. The method can further include isolating the sperm from the provided sample, e.g., by centrifugation. In one embodiment, the sperm sample is at a temperature of between about 27° C. and about 38° C., preferably about 37° C., prior to cooling. The sperm sample can be obtained from a mammal, e.g., a goat, a cow, a sheep, a rabbit, a pig, or a mouse, preferably a goat or a rabbit. In a preferred embodiment, the mammal can be a transgenic mammal, e.g., a mammal containing a transgene encoding a polypeptide. The polypeptide can be any protein, whose expression is desired in a transgenic mammal, including any of: α-1 proteinase inhibitor, alkaline phosphatase, angiogenin, extracellular superoxide dismutase, fibrinogen, glucocerebrosidase, glutamate decarboxylase, human serum albumin, myelin basic protein, proinsulin, soluble CD4, lactofenin, lactoglobulin, lysozyme, lactalbumin, erythrpoietin, tissue plasminogen activator, human growth factor, antithrombin III, insulin, prolactin, and α1-antitrypsin. The transgene can further include a promoter, e.g., a milk specific promoter. The milk specific promoter can be any of: a casein, a whey acid protein, an α-lactalbumin, a β-lactoglobin, or a lactoferrin promoter.

The method includes providing the sample to be cooled in a cryoprotectant buffer. In a one embodiment, the cryoprotectant buffer includes glycerol, e.g., about 5% to 10% glycerol, preferably about 7% glycerol. In another preferred embodiment, the cryoprotectant buffer lacks glycerol. The cryoprotectant buffer can include egg yolk, e.g., about 10% to about 30% egg yolk, e.g. about 15% to 25% egg yolk, preferably 20% egg yolk. The first cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In one embodiment, the sperm sample is cooled to a first temperature of about 1° C. to about 8° C., more preferably about 5° C. In a preferred embodiment, the sperm sample is cooled to the first temperature at a rate of about 0.2° C. to about 0.5° C. per minute, preferably about 0.5° C. per minute. In another preferred embodiment, the sperm sample is cooled over the course of about 1.5 hours to about 4 hours, preferably about 1.5 hours. The sperm sample can be maintained at the first temperature for a period of time, e.g., between about 4 hours and about 21 hours, preferably about 4 hours.

In a preferred embodiment, when a cryprotectant buffer lacking glycerol is added prior to cooling, a second cryoprotectant buffer can be added to the cooled sperm sample while it is at the first temperature. The second cryoprotectant buffer comprises glycerol, at a concentration such that after addition the sample, the sample has a glycerol concentration of about 5% to 10% glycerol, preferably about 7% glycerol. The second cryoprotectant buffer can include egg yolk, e.g., about 10% to about 30% egg yolk, e.g. about 15% to 25% egg yolk, preferably 20% egg yolk. The second cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In one embodiment, the cooled sperm sample is frozen at a second temperature of about −80° C. In another preferred embodiment, the frozen sperm sample is maintained at the second temperature for about 7 minutes to 20 minutes, preferably for about 10 minutes to about 18 minutes, more preferably for about 15 minutes.

In one embodiment, the frozen sperm sample is stored at a third temperature of about −180° C. to about −220° e.g. −196° C., e.g., in liquid nitrogen. The sperm sample can be maintained at the third temperature until further use. In another embodiment, the frozen sperm sample is thawed from the third temperature. Preferably, the sample is thawed at about 27° C. to about 38° C., for about 1 minute to about 5 minutes, preferably for about 1.5 minutes. In one embodiment, the percentage of viable sperm after thawing is about 20%, 30%, 40%, 50%, 55%, 60%, 65%, 70%, 75%, 80%, 85%, 90%, 95%, 98%, 99%, or 100%.

Another aspect of the invention features a method of providing sperm. The method includes: providing a sample comprising sperm; isolating sperm from the sample; combining the isolated sperm with a first cryoprotectant buffer; cooling the sperm to a first temperature of about 2° C. to about 8° C., e.g., about 5° C., at a rate of about 0.2° C. to 0.5° C. per minute, preferably of about 0.5° C., to produce cooled sperm; adding a second cryoprotectant buffer; maintaining the cooled sperm at the first temperature for a duration of about 4 hours to about 21 hours, preferably about 4 hours; freezing the cooled sperm at a second temperature of about −60° C. to about −90° C. for a time of between about 10 minutes to about 15 minutes, e.g., about 15 minutes; and storing the frozen sperm at a third temperature of about −180° to about −220° C., preferably −196° C., e.g., in liquid nitrogen. The sample can be maintained at the third temperature until further use.

In one embodiment, the method includes providing a sample of semen, e.g., semen obtained from live animals. In another embodiment, the sperm sample is obtained by extraction from the epididymis, e.g., at necropsy. The method can further include isolating the sperm from the provided sample, e.g., by centrifugation. In one embodiment, the sperm sample is at a temperature of between about 27° C. and about 38° C., preferably about 37° C., prior to cooling. The sperm sample can be obtained from a mammal, e.g., a goat, a cow, a sheep, a rabbit, a pig, or a mouse, preferably a goat or a rabbit. In a preferred embodiment, the mammal can be a transgenic mammal, e.g., a mammal containing a transgene encoding a polypeptide. The polypeptide can be any protein, whose expression is desired in a transgenic mammal, including any of: α-1 proteinase inhibitor, alkaline phosphatase, angiogenin, extracellular superoxide dismutase, fibrinogen, glucocerebrosidase, glutamate decarboxylase, human serum albumin, myelin basic protein, proinsulin, soluble CD4, lactoferrin, lactoglobulin, lysozyme, lactalbumin, erythrpoietin, tissue plasminogen activator, human growth factor, antithrombin III, insulin, prolactin, and α1-antitrypsin. The transgene can further include a promoter, e.g., a milk specific promoter. The milk specific promoter can be any of: a casein, a whey acid protein, an α-lactalbumin, a β-lactoglobin, or a lactoferrin promoter.

In a preferred embodiment, the first cryoprotectant buffer lacks glycerol. In a preferred embodiment, the sperm to be cooled are combined with in a cryoprotectant buffer that includes egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In a preferred embodiment, the second cryoprotectant buffer comprises glycerol, e.g., about 5% to 10% glycerol, preferably 7% glycerol. Preferably, the cryoprotectant buffer further includes egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In one embodiment, the sperm sample is cooled to the first temperature over the course of about 1.5 hours to about 4 hours, preferably 1.5 hours.

In a preferred embodiment, the frozen sperm sample is thawed, e.g., at between about 27° C. to about 38° C., for about 1 minute to about 5 minutes, preferably for about 1.5 minutes. In one embodiment, the percentage of viable sperm after thawing is about 20%, 30%, 40%, 50%, 55%, 60%, 65%, 70%, 75%, 80%, 85%, 90%, 95%, 98%, 99%, or 100%.

Another aspect of the invention features a method for making an animal, e.g., a mammal. The method includes fertilizing an oocyte with sperm provided by any of the methods described herein. In one embodiment, the oocyte is fertilized in vivo. For example, the thawed sperm is deposited intra-cervically or in utero. In another embodiment, the oocyte is fertilized in vitro. In a preferred embodiment, the oocyte utilized for in vitro fertilization can be matured in vitro or in vivo.

In one embodiment, the method includes fertilizing an oocyte with sperm obtained from a mammal. The mammal can be a goat, a cow, a sheep, a rabbit, a pig, or a mouse. Preferably, the mammal is a goat or a rabbit. In a preferred embodiment, the mammal is a transgenic mammal, e.g., a transgenic mammal containing a transgene encoding a polypeptide. The polypeptide can be any protein, whose expression is desired in a transgenic mammal, including any of: α-1 proteinase inhibitor, alkaline phosphatase, angiogenin, extracellular superoxide dismutase, fibrinogen, glucocerebrosidase, glutamate decarboxylase, human serum albumin, myelin basic protein, proinsulin, soluble CD4, lactoferrin, lactoglobulin, lysozyme, lactalbumin, erythrpoietin, tissue plasminogen activator, human growth factor, antithrombin III, insulin, prolactin, and α1-antitrypsin. The transgene can further include a promoter, e.g., a milk specific promoter. The milk specific promoter can be any of: a casein, a whey acid protein, an α-lactalbumin, a β-lactoglobin, or a lactoferrin promoter.

Another aspect of the invention features an animal, for example an animal derived from an oocyte fertilized by sperm prepared by any of the methods described herein.

Another aspect of the invention features a sample of preserved sperm that has been treated by any of the methods of this invention.

Another aspect of the invention features a kit for cryopreserving sperm which includes a cryoprotectant buffer. The kit further includes instructions for preserving sperm.

In a preferred embodiment, the cryoprotectant buffer can include glycerol, e.g., between about 5% and 10% glycerol, preferably 7% glycerol. In another preferred embodiment, the cryoprotectant buffer can lack glycerol. The cryoprotectant buffer can include egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

In a preferred embodiment, the instructions include a protocol detailing any of the methods described herein. In another embodiment, the kit can further include sterile plastic straws; and a rack for the straws. In another embodiment, the kit further includes stains for assaying sperm viability, preferably with instructions for their usage.

Another aspect of the invention features a kit for making an animal, e.g., a mammal. The kit includes a cryoprotectant buffer, instructions for preserving sperm by any of the methods described herein, and instructions for fertilizing an oocyte with preserved sperm.

In a preferred embodiment, the cryoprotectant buffer can include glycerol, e.g., between about 5% and 10% glycerol, preferably 7% glycerol. In another preferred embodiment, the cryoprotectant buffer can lack glycerol. The cryoprotectant buffer can include egg yolk, e.g., about 10% to about 30% egg yolk, e.g. about 15% to 25% egg yolk, preferably 20% egg yolk. The cryoprotectant buffer can further include one or more of: fructose, e.g. fructose at a concentration of about 1% weight to volume; citric acid, e.g., citric acid at a concentration of about 1.5% weight to volume; Tris buffer; an antibiotic compound, for example tylosin, gentamicin, lincospectin, and/or spectinomycin.

Another aspect of the invention features a kit for making an animal. The kit includes sperm preserved by the methods described herein and instructions for fertilizing an oocyte with the preserved sperm.

In one embodiment, the method includes fertilizing an oocyte with sperm obtained from a mammal. The mammal can be a goat, a cow, a sheep, a rabbit, a pig, or a mouse. Preferably, the mammal is a goat or a rabbit. In a preferred embodiment, the mammal is a transgenic mammal, e.g., a transgenic mammal containing a transgene encoding a polypeptide. The polypeptide can be any protein, whose expression is desired in a transgenic mammal, including any of: α-1 proteinase inhibitor, alkaline phosphatase, angiogenin, extracellular superoxide dismutase, fibrinogen, glucocerebrosidase, glutamate decarboxylase, human serum albumin, myelin basic protein, proinsulin, soluble CD4, lactoferrin, lactoglobulin, lysozyme, lactalbumin, erythrpoietin, tissue plasminogen activator, human growth factor, antithrombin III, insulin, prolactin, and α1-antitrypsin. The transgene can further include a promoter, e.g., a milk specific promoter. The milk specific promoter can be any of: a casein, a whey acid protein, an α-lactalbumin, a β-lactoglobin, or a lactoferrin promoter.

As used herein, the term “transgenic sequence” refers to a nucleic acid sequence (e.g., encoding one or more human proteins), which is inserted by artifice into a cell. The transgenic sequence, also referred to herein as a transgene, becomes part of the genome of an animal which develops in whole or in part from that cell. In embodiments of the invention, the transgenic sequence is integrated into the chromosomal genome. If the transgenic sequence is integrated into the genome it results, merely by virtue of its insertion, in a change in the nucleic acid sequence of the genome into which it is inserted. A transgenic sequence can be partly or entirely species-heterologous, i.e., the transgenic sequence, or a portion thereof, can be from a species which is different from the cell into which it is introduced. A transgenic sequence can be partly or entirely species-homologous, i.e., the transgenic sequence, or a portion thereof, can be from the same species as is the cell into which it is introduced. If a transgenic sequence is homologous (in the sequence sense or in the species-homologous sense) to an endogenous gene of the cell into which it is introduced, then the transgenic sequence, preferably, has one or more of the following characteristics: it is designed for insertion, or is inserted, into the cell's genome in such a way as to alter the sequence of the genome of the cell into which it is inserted (e.g., it is inserted at a location which differs from that of the endogenous gene or its insertion results in a change in the sequence of the endogenous gene); it includes a mutation, e.g., a mutation which results in misexpression of the transgenic sequence; by virtue of its insertion, it can result in misexpression of the gene into which it is inserted, e.g., the insertion can result in a knockout of the gene into which it is inserted. A transgenic sequence can include one or more transcriptional regulatory sequences and any other nucleic acid sequences, such as introns, that may be necessary for a desired level or pattern of expression of a selected nucleic acid, all operably linked to the selected nucleic acid. The transgenic sequence can include an enhancer sequence and or sequences which allow for secretion.

As used herein, the term “transgenic cell” refers to a cell containing a transgene.

As used herein, a “transgenic animal” is a non-human animal in which one or more, and preferably essentially all, of the cells of the animal contain a heterologous nucleic acid introduced by way of human intervention, such as by transgenic techniques known in the art. The transgene can be introduced into the cell, directly or indirectly by introduction into a precursor of the cell, by way of deliberate genetic manipulation, such as by microinjection or by infection with a recombinant virus.

Mammals are defined herein as all animals, excluding humans, which have mammary glands and produce milk.

As used herein, “semen” refers to the ejaculate of a male animal, which contains sperm.

As used herein, “epididymal sperm” refers to sperm obtained by surgical dissection of the epididymis of the testes.

As used herein, “cryoprotectant” refers to an agent which can reduce the affects of freezing, thawing, and/or storage at temperatures below freezing. Examples of cryoprotectants include, e.g., glycerol and ethylene glycol.

As used herein, “extending buffer” refers to a solution containing agents that enhance sperm viability, motility, and/or fertility, during incubation, freezing, storage, and/or thawing as compared to sperm viability, motility, and/or fertility without extending buffer.

“Artificial insemination” is defined as the process of fertilizing female animals by manual injection or application of sperm. In such a procedure, male animals are not required at the time on insemination, as the sperm is obtained from them previously.

The percent of viable sperm can be determined by dividing the number of viable sperm observed by the number of total sperm observed in the same sample. This is also referred to herein as the live/dead ratio.

The invention provides several benefits including the maintenance and preservation of fertile male gametes that may be acquired from rare and valuable genetic stocks, such as endangered species, transgenic animals, and individuals. For example, the invention provides for the preservation of sperm, e.g., from male animals, that unexpectedly die or require euthanization. This method can be of value in preserving endangered species whose contribution to biodiversity cannot yet be assessed. Preservation of sperm is also useful when the species in question has limited or seasonal breeding cycles. This invention generally facilitates the expansion and maintenance of animals with consistent genetic composition over time.

The current invention also provides several benefits with regards to transgenic animals. Transgenic animals are expensive commercial investments that are sometimes difficult and costly to create. For example, due to the inherent randomness and low frequency of the insertion of the transgene into genomes, individual founder transgenic animals can carry the transgene in only a fraction of cells, e.g. they are mosaics. In addition, they can express a transgenic protein in their milk at varying concentrations. Thus, the selection and preservation of spermatozoa from highly expressing individuals provides long-term security for the initial investment into generating the transgenic animal, as well as cost savings by obviating the need to screen and cull progeny.

Other features and advantages of the invention will be apparent from the following detailed description, and from the claims.

### DETAILED DESCRIPTION OF THE INVENTION

Detailed methods for cryopreserving sperm are described herein and in the section entitled “Examples” below.

The invention provides methods of preserving sperm, e.g., sperm from transgenic mammals, which can later be used to produce an animal, e.g., a transgenic mammal. Several steps can be used in the disclosed methods including: obtaining samples containing sperm, assaying sperm viability, isolating sperm, cryopreserving the sperm sample, artificially inseminating a recipient animal or providing an embryo by in vitro procedures, including in vitro fertilization of in vivo or in vitro matured oocytes.

This invention is further illustrated by the following examples, which in no way should be construed as being further limiting. The contents of all cited references (including literature references, issued patents, published patent applications, and co-pending patent applications) cited throughout this application are hereby expressly incorporated by reference.

Obtaining Sperm Samples

A sample comprising sperm to be preserved can be obtained by several methods. The term “sperm,” as used herein, refers to mature mate gametes. The terms “sperm” and “spermatozoa” are used interchangeably herein. Methods of obtaining a sperm sample can include obtaining semen from male animals or by extraction of sperm from the epididymis.

Semen can be obtained from an animal by stimulation with an artificial vagina. For example an artificial vagina can be used as follows.

Prior to sample collection, a water bath is equilibrated to 37° C., and the extender solution (Continental Plastics Corp., Delavan, Wis.) containing 7% glycerol, 2.42% Tris buffer, 1.38% citric acid, 1% fructose, antibiotics (5.5 mg Tylosin, 27.5 mg gentamicin, 16.5 mg lincospectin, and 33.0 mg per 100 ml) and 20% volume to volume egg yolk (specific pathogen free, SPAFAS, Norwich Conn.), is equilibrated to this temperature. A thermos with a thermometer is set up with 35-39° C. water for holding and transporting the freshly collected sample. An artificial vagina is also prepared. Preferably, the artificial vagina is broken down into its component pieces and thoroughly cleaned with hot water and a 10% Nolvasan solution prior to use. All pieces are then rinsed with RO/DI water and dried. The type of artificial vagina which can be used is comprised of a firm rubber outer ring structure, approximately 6-10 inches in length, and an inflatable inner rubber lining. This inner lining is filled with warm water then inflated with air to provide adequate pressure. Another inner lining, with a tapered conical open at one is placed within the artificial vagina apparatus. A moderate amount of sterile gynecological lubricant is applied to one end and a 15 ml sterile conical tube is inserted at the other.

Bucks can be examined to ensure they are in good health. An appropriate teaser is chosen. The teaser can be an ovariectomized doe, that has been primed approximately twenty four hours earlier with exogenous estrogen, a teaser that on the day of collection is in heat, or any animal that will provide enough stimulus (i.e. another buck). Semen is collected using an artificial vagina and a teaser female to simulate the buck. Samples are immediately mixed with equilibrated extender (Continental Plastics Corp., Delavan, Wis.) containing 7% glycerol, 2.42% Tris buffer, 1.38% citric acid, 1% fructose, antibiotics (5.5 mg Tylosin, 27.5 mg gentamicin, 16.5 mg lincospectin, and 33.0 mg per 100 ml) and 20% volume to volume egg yolk (specific pathogen free, SPAFAS, Norwich Conn.). The samples are immediately transported back to the laboratory for analysis and preservation.

Sperm, e.g., epididymal sperm, can be obtained directly from the epididymis of the animal. This method can be used to obtain sperm from both live and dead animals. Methods for extracting sperm from the epididymis are known in the art, see for example Sharma et al. (1997) Fertil Steril. 68:626-631, and are also set forth in more detail in the examples below.

Assaying Sperm Viability

The sperm sample obtained can then be analyzed to determine the condition of the sperm by, e.g., wave motion analysis, motility assays, and viability counts.

For example, a gross microscopic analysis of the semen can be conducted by analyzing wave motion under low magnification (10×) and ascribing a score for motion from 0-5, with 0 being no wave motion and 5 being rapid wave motion with eddies. Secondly, under higher magnification (40×), the number of motile sperm can be counted and scored as a percentage of total sperm. This percentage is later multiplied by the concentration/count to determine the number of visibly viable sperm. Preferably, the sample is of high enough quality to cryopreserve. For example, sperm having at least about 40% motility can be used. Sperm concentration can be determined by various procedures: a microcuvette containing semen diluted 1:10 with 0.9% saline is assayed in a Spermacue photometer; or a series of dilutions (1:1000) of the sperm are made and counted with a hemocytometer.

The percentage of viable sperm ratio can be determined by placing a 15 μl drop of extended sample of sperm on a microscope slide with a 15 μl drop of a Live/Dead stain (Morphology Stain, Lane Manufacturing, Inc., Denver Co.). A thin smear is prepared after mixing the two drops. The sample is air dried, and then 200 individual sperm are counted by staining with the vital dye under the microscope with a 100× oil immersion lens.

Lastly, a sperm's integrity can be assayed by observation of the sperm's acrosomal cap and tail morphology using the Spermac stain. Another microscope slide is prepared with a 15 μl drop of sperm, air dried, and then stained with Spermac following the manufacturer's specification. The overall quality and morphology of the sample is determined by scoring acrosomal caps as intact or non-intact and by counting the number normal tails per 200 individual sperm.

Isolating Sperm

Sperm can be optionally isolated from the provided sample. For example, after the addition of extender buffer to a 10 ml volume, the sample can be centrifuged for 15 minutes at approximately 1500 rpm's (500-600× g) or until the sperm is adequately separated. The supernatant is decanted. Samples of adequate quality are then diluted with extender solution to the appropriate amount of sperm needed per straw. Although 0.5 ml straws are usually used, 0.25 ml straws can be used when needed. The amount of extender to add can vary between samples. The amount of extender can be adjusted to ensure a sperm count of 100-150 million viable sperm per straw, preferably 150 million.

Two types of extender solution can be used. If a one-step extender solution is used, the entire volume of extender can be added at this stage. The one-step extender contains glycerol. If a two-step extender solution is used, a portion of the final volume of extender, e.g., about half the volume, can be added at this juncture. The first part of the two part extender, Part A, lacks glycerol. The second part, Part B, contains glycerol and is, preferably, added after the sperm are cooled to the first temperature. Part A extender can include: egg yolk, Part A buffer concentrate, and/or an antibiotic concentrate. Part B extender can include: egg yolk, Part B buffer concentrate, and/or 2 an antibiotic concentrate.

Part A extender and Part B extender can be prepared, for example, as follows. Both antibiotics and egg yolk are supplemented into the Part A extender and Part B extender prior to use. A volume of eggs can be prepared by washing the eggs in a chlorhexidine solution and dried with paper towels. Each egg is cracked open, taking care not to rupture the yolk sac. The albumin is removed from the yolk by separating the yolk and the albumin with the egg shell. The yolk sac is poured onto gauze that has been laid over a beaker. The yolk is punctured, which allows the yolk to flow through the gauze. Enough egg yolks are processed to make a 20% (v/v) solution of yolk in each extender, Part A and Part B. Each part can be made up separately. For each Part A and Part B, the extender concentrate is poured into a graduated cylinder, the egg yolk and antibiotics are added to the extender, and the solution made up to 500 mls with sterile water. Preferably, the concentrate, egg yolk and antibiotics are added at the following volumes. For one liter of Part A extender, 200 mls egg yolk, 340 mls Part A concentrate, 20 mls reconstituted antibiotic solution are added, and then sterile water added to a final volume of 1 liter. For one liter of Part B extender, 200 mls egg yolk, 340 mls Part B concentrate, 2 mls reconstituted antibiotic solution are added, and then sterile water added to a final volume of 1 liter. Forty-five ml aliquots of extender can then be poured into steril 50 ml centrifuge tubes which can be labeled, dated and stored frozen at −20° C.

Cryopreserving Sperm

Once the semen has been extended to the proper dilution, it is ready for cryopreservation. Preferably, the sample is maintained at a temperature of about 37° C. until this point. The cryopreservation process can be started by placing a tube which contains the extended semen into a beaker containing water at approximately 37° C. This configuration is placed in a refrigerator. This initial cooling preferably lowers the sample temperature to 5° C. (+/−2° C.) in no less than 1.5 hours. During the cooling process the sample can be mixed, the temperature can be monitored, and the rate of cooling can be determined.

If the two-step extender solution is utilized, then part B of the extender can be added when the sample reaches approximately 5° C.

The sample can be maintained at approximately 0-5° C. (+/−2° C.) for a minimum of about 4 hours and no longer than about 21 hours prior to freezing. Preferably, the sample is stored inside a refrigerator maintained at about 5° C. (+/−2° C.) for about 4 hours.

After this equilibration period, the samples can be transferred into plastic straws, pre-cooled to about 5° C.(+/−2° C.). The straws are filled, sealed with a plastic plug or heat sealed, and placed on the straw rack in a bed of ice until all are finished. The rack of straws can then be placed inside a −80° C. freezer. Preferably, the straws are maintained in the −80° C. freezer for approximately 15-20 minutes. Just prior to being placed into liquid nitrogen, the straws are placed inside canes and goblets pre-cooled to −80° C.

Once placed in liquid nitrogen, the straws can be stored in nitrogen tanks. Within 3 to days following cryopreservation, one straw from each sperm sample can be analyzed. The frozen straws are thawed for 90 seconds in 37° C. water. Then, the percentage of viable sperm, and the integrity of acrosomal caps and tail morphology can be determined as described above.

Artificially Inseminating a Recipient Animal

In one embodiment of the invention, cryopreserved sperm can be utilized to artificially inseminate female recipients. Estrus synchronization in recipients can be induced by 6 mg norgestomet ear implants (Syncromate-B, Rhone-Meriuex, Athens Ga.). On Day 13 after insertion of the implant, the animals are given single non-superovulatory injection (400 I.U.) of pregnant-mare serum gonadotropin (PMSG, Calbiochem-Novabiochem Corp., La Jolla Calif.). Recipient females are mated to vasectomized males to ensure estrus synchrony (Selgrath, et al., Theriogenology, 1990. pp. 1195-1205). Sperm can then be thawed as described above and used to inseminate the recipient females following methods commonly practiced by those skilled in the art.

Providing an Embryo

In another embodiment, oocytes can be collected from female animals for in vitro fertilization with cryopreserved sperm. As described above, norgestomet ear implants can be used to synchronize estrus. A single injection of prostaglandin (PGF2α) (Upjohn, US) is administered on day 7. Starting on day 12, the females are administered FSH (Folltropin-V, Vetrepharm, Canada) twice daily for four consecutive days. The norgestomet ear implant is removed on day 14. Twenty-four hours after implant removal, the females are mated several times to vasectomized males over a 48 hour period. Following the final FSH injection, the females are injected with a single dose of GnRH (Rhone-Meriuex, Athens Ga.). Oocytes are recovered surgically from the female donors by mid-ventral laparotomy approximately 18 to 24 hours following the last mating. Oocytes are flushed from the oviduct with Ca⁺⁺/Mg⁺⁺-free PBS (phosphate-buffered saline) prewarmed at 37° C. Recovered oocytes are cultured in equilibrated M199 with 10%FBS supplemented with 2 mM L-glutamine, 100 U/ml penicillin and 100 μg/ml streptomycin.

The recovered oocytes can then be combined with thawed sperm following methods commonly practiced in the art. Sperm was thawed and purified using a 90%-45% Percoll gradient and fertilization was performed in 50 μl drops of B-O media supplemented with 20% FBS, 7.7 mM calcium lactate, 100 U/ml penicillin and 100 μg/ml streptomycin under oil for 18 hours incubated in 5% CO₂ at 38° C. In vitro culture was performed in M199 plus 10% FBS with primary goat oviductal epithelial cell co-cultures. An embryo can be maintained in culture until at least first-cleavage (2-cell stage) and up to blastocyst stage. Preferably the embryos are transferred at the 2 or 4-cell stage. Various culture media for embryo development are known in the art as are methods for transferring an embryo to a recipient, for example see Ebert et al. (1994) Bio/Technology 12:699.

The invention is further illustrated by the following examples which should in no way be construed as limiting.

### EXAMPLE 1

Thirty-two bucks, consisting of 3 breeds (Alpine, Saanen, and Toggenburg) ranging in age from 13 days to 7 years were utilized. The treatment of these animals followed the Institutional Animal Care and Use Committee (IACUC) approved protocol and followed regulations stated in the Animal Welfare Act (AWA). Animals were euthanized with an intravenous barbiturate overdose. The testes were removed within about 5 to about 10 minutes from the scrotal sac, and placed in a 38° C. incubator. Testes were processed individuals. Using a sterile scalpel, the parietal tunic was removed leaving the tail of the epididymis exposed. A small lateral incision was made along the tail of the epididymis to open the convoluted tubules. Slight pressure was applied to the tail, allowing small droplets of sperm to form. The droplets were pipetted into equilibrated extender (Continental Plastic Corp., Delavan Wis.) consisting of 20% v/v egg yolk (standard-pathogen free, SPAFAS, Norwich, Conn.), 7% glycerol, 2.42% Tris buffer, 1% fructose, 1.38% citric acid, 5.5 mg Tylosin, 27.5 mg Gentamicin, 16.5 mg Lincospectin, and 33 mg spectinomycin per 100 ml. This process was repeated until the sperm in the epididymal tail was fully extracted. The sperm was pooled from both testes. Epididymal sperm was successfully collected and cryopreserved from the 25 bucks in which epididymal sperm was present. The average age of the bucks producing sperm was 2.1 years with a range of 4 months to 7 years. The seven bucks that did not have sperm were all under the age of 4 months.

15 μl of material was used analyze sperm motility and wave motion. Each sample was assigned a motion score for individual sperm from 0-5 (0=no movement, and 5=rapid linear movement). The percentage of live/dead sperm was determined by placing a 15 μl sample and 15 μl of Morphology Stain™ (Society for Theriogenology, Hastings, Nebr.) on a slide, mixing the two drops together, and preparing a thin smear. Under an 100× oil immersion lens, a random count of 200 sperm in each sample was performed. Acrosomal integrity was similarly determining using the Spermac® stain (Minitube of America, Verona, Wis.). The mean number of sperm extracted was 3.8×10⁹±2.0×10⁹ with a range of 1.1×10⁹ to 12.3×10⁹. The average live/dead ratio of epididymal sperm as 92% with a range from 63% to 97%. The average post-thaw live-dead ratio was 83% with a range of 32% to 93%. In addition, 84% of samples had intact acrosomes after post-thaw. These data are in Table 1.

TABLE 1
Analysis of sperm motility, live/dead ratio, and sperm number
for epididymal collections

| Goat | | | | Post- | Total # |
|---|---|---|---|---|---|
| Age | | Sperm | % | Thaw | of Sperm |
| (months) | N = | motility | viable | % viable | 10⁹/ml |
| 4-6 | 1 | 5 | 92.0± | 80.0± | 1.3± |
| 6-18 | 11 | 5 | 94.0± | 87.0± | 4.3± |
| 18- | 13 | 5 | 90.0± | 79.0± | 2.4± |
| 4-84 | 25 | 5 | 92.0± | 83.0± | 3.7± |

Values are expressed as mean ± standard deviation.

Combined semen samples were centrifuged and resuspended to 300×10⁶ sperm per ml with fresh equilibrated extender. Samples were placed in a 37° C. water bath, refrigerated, and cooled to 5° C., over 1.5 hours, at a rate of 0.5° C. per minute. The sample was maintained at this temperature for a minimum of 4 hours and a maximum of 21 hours. Sperm samples were then loaded into 0.5 ml straws, placed into a −80° C. freezer for about 10 to about 15 minutes, and then plunged into liquid nitrogen. After cryostorage for at least three days, one straw was thawed at 37° C. for 2 minutes to determine the post-thaw live/dead percentage and acrosomal integrity as described above. Oocytes were aspirated from ovaries obtained from does at necropsy, out of season for the Northern hemisphere, and matured in vitro for 18-24 hours in M199 (GibcoBRL) supplemented with 10% fetal bovine serum, FSH 5.0 U/ml, LH 5.0 U/ml, β-estradiol 1 μg/ml, and penicillin-streptomycin. Sperm was thawed and purified using a 90%-45% Percoll gradient and fertilization was performed in 50 μl drops of B-O media supplemented with 20% FBS, 7.7 mM calcium lactate, 100 U/ml penicillin and 100 μg/ml streptomycin under oil for 18 hours incubated in 5% CO₂ at 38° C. In vitro culture was performed in M199 plus 10% FBS with primary goat oviductal epithelial cell co-cultures.

In vitro fertilization using cryopreserved and subsequently thawed epididymal sperm resulted in 40% of oocytes exhibiting cleavage and 6% developing to the blastocyst stage (Table 2). In comparison, in vitro fertilization using ejaculated sperm resulted in 37% of oocytes cleaving and 4% developing to the blastocyst stage.

Animals used for artificial insemination were synchronized by using a progesterone implant (Synchromate-B, Rhone Meriux, Athens Ga.) on day 0. On day 7, 5 mg of PGF2 (Pharmacia & Upjohn) was administered followed by 300-400 IU IM of PMSG (Calbiochem-Novabiochem) on day 14. The progesterone implant was removed on day 14 and breeding to vasectomized bucks was performed on days 15-16. Does where checked for signs of heat with a vasectomized buck. Approximately 12 hours after standing heat, does were inseminated once with one straw of thawed sperm. The technique used was either intra-cervical or intra-uterine deposition. Artificial insemination was performed on 21 does. This process was repeated every 12 hours until the doe was no longer in heat. Does were ultrasounded between days 32 and 36 and again between days 55 to 60. Does were monitored daily from day 145. Kids were removed at birth and the doe was placed into normal milk production. The artificial insemination of 21 does resulted in one pregnancy (4%) of which one healthy kid was born.

TABLE 2
Results of caprine oocytes in vitro fertilization
using thawed sperm.

| Sperm | | # of | Cleaved | Morula | Blasts. |
|---|---|---|---|---|---|
| Sample | N = | Oocytes | (%) | (%) | (%) |
| Epididymal | 3 | 168 | 67/168 | 32/168 | 10/168 |
| | | | (40%) | (19%) | (6%) |
| Ejaculated | 3 | 169 | 63/169 | 29/169 | 6/169 |
| | | | (37%) | (17%) | (4%) |

The successful cryopreservation of epididymal sperm from the testes of valuable animals allows its genetic contribution to be passed on when that animal unexpectedly dies or needs to be euthanized. Potentially such a procedure could even be performed a few hours after death. In the present study, 25 of 25 animals where epididymal semen was extracted resulted in cryopreserved sperm. This is consistent with published works on epididymal extraction in other species (Foote and Igboeli (1968) J Diary Sci 10:1703-5; Pauffler et al.(1968) J Reproduction Fertil 17:125-137).

Epididymal sperm was placed into a controlled caprine in vitro embryo production system which resulted in the cleavage and development of blastocysts. No developing embryos were transferred to recipient. Sperm used for artificial insemination resulted in a pregnancy which has also been reported in other species (Foote and Igboeli, supra; Sharma et al. supra).

Several factors potentially affecting the quantity and quality of sperm extracted from the epididymis may include the age of the animal and the time of year. Goats are seasonal breeders; therefore, the amount of sperm extracted from the epididymis during the nonbreeding season may be less then that extracted during the breeding season. The minimum age when sperm can be collected is 4 months during the breeding season. One possible way to decrease the age in which sperm could be extracted may be to have the buck interact with estrous does during the breeding season. This may help stimulate the reproductive system and initiate sperm production earlier through environmental factors.

In conclusion epididymal sperm may be cryopreserved from goats at necropsy in good quantity and quality. This sperm may be used for in vitro development or artificial insemination to propagate valuable genetics. Factors such as optimal breeding season and decreased age to semen production may have a beneficial effect. By increasing the quantity of the semen extracted from the testes, a greater yield of sperm may result. Further work needs to be performed to investigate these theories in parallel with optimizing the use of epididymal sperm for artificial insemination.

### EXAMPLE 2

Sperm from transgenic male rabbits were collected using an artificial vagina. Samples were diluted with Part A extender which lacks glycerol and transported back to the laboratory in a thermos at a temperature of about 37° C. Samples were treated as in Example 1 with the following exception. Straws were loaded with 20 million sperm per straw. After sample cooling to 4° C., Part B extender which contains glycerol was added. Then, the samples were frozen to −80° C. as specified in example 1, the samples were stored in liquid nitrogen for one month. Four Dutch rabbits were synchronized with hormone, follicle stimulating hormone and human chorionic gonadotrophin. Sperm samples were thawed by placement in a water bath at 37° C. for 90 seconds. Samples were then used to artificially inseminate the synchronized females. Eleven progeny were born from two mothers inseminated with the samples.

All patents and references cited herein are incorporated in their entirety by reference.

Other embodiments are within the following claims.

What is claimed is:

1. A method of providing sperm comprising:
a) cooling a sample which includes sperm to a first temperature sufficient to protect sperm from glycerol toxicity, at a rate sufficiently slow that the metabolic rate of sperm is decreased, to provide a cooled sperm sample;
b) adding a solution comprising glycerol to the cooled sperm sample; and
c) freezing said cooled sperm sample to second temperature for a sufficient period of time to equilibrate glycerol and sperm to thereby provide a frozen sperm sample, such that the sperm is preserved.

2. The method of claim 1, further comprising: providing a sperm sample to be cooled in a cryoprotectant buffer lacking glycerol.

3. The method of claim 1, wherein the cryoprotectant buffer comprises about 10% to about 30% egg yolk.

4. The method of claim 1, wherein the concentration of glycerol in the sample after the addition of the glycerol solution is about 5% to about 10% glycerol.

5. The method of claim 1, wherein the sperm sample is obtained from a mammal.

6. The method of claim 1, wherein the first temperature is between about 0° C. to about 10° C.

7. The method of claim 1, wherein the sperm sample is maintained at the first temperature for between about 4 hours to about 21 hours.

8. The method of claim 1, wherein the sperm is cooled at a rate of between about 0.2° C. to about 0.5° C. per minute to the first temperature.

9. The method of claim 1, wherein the sperm is cooled to the first temperature over the course of about 1.5 to about 4 hours.

10. The method of claim 1, wherein the second temperature is between about −40° C. to about −100° C.

11. The method of claim 1, wherein the sample is maintained at the second temperature for between about 7 minutes to about 20 minutes.

12. The method of claim 1, wherein the method further comprises storing the frozen sperm sample at a third temperature of about −190° C. to about −200° C.

13. A method of preserving sperm comprising:
a) combining sperm with a first cryoprotectant buffer;
b) cooling said sperm to a first temperature between about 2° C. to about 10° C. at a rate sufficiently slow that the metabolic rate of sperm is decreased to produce cooled sperm;
c) freezing the cooled sperm at a second temperature between about −60° C. to about −90° C.; and
d) storing the frozen sperm in liquid nitrogen.

14. The method of claim 13, wherein the first cryoprotectant buffer comprises about 5% to about 10% glycerol.

15. The method of claim 13, wherein the first cryoprotectant buffer lacks glycerol.

16. The method of claim 13, wherein the sperm sample is obtained from a mammal.

17. The method of claim 13, wherein the sperm sample is maintained at the first temperature for between about 4 hours to about 21 hours.

18. The method of claim 13, wherein the sperm is cooled at a rate of between about 0.2° C. to about 0.5° C. per minute to the first temperature.

19. The method of claim 13, wherein the sperm is cooled to the first temperature over the course of about 1.5 hours to about 4 hours.

20. The method of claim 15, wherein a second cryoprotectant buffer is added to the sample after the sperm is cooled to the first temperature, and before the sperm is further cooled to the second temperature.

21. The method of claim 20 wherein the second cryoprotectant buffer comprises about 5% to about 10% glycerol.

22. The method of claim 13, wherein the second temperature is about −80°.

23. The method of claim 13, wherein the sample is maintained at the second temperature for between about 7 minutes to about 20 minutes.

24. A method of providing sperm comprising:
a) providing a sample comprising sperm;
b) isolating sperm from the sample;
c) combining said isolated sperm with a first cryoprotectant buffer which lacks glycerol;
d) cooling said sperm to a first temperature of about 2° C. to about 8° C. at a rate of about 0.2° C. to 0.5° C. per minute to produce cooled sperm;
e) adding a second cryoprotectant buffer which contains glycerol;
f) maintaining the cooled sperm at the first temperature for a duration of about 4 hours to about 21 hours;
g) freezing the cooled sperm at a second temperature of about −60° C. to about −90° C. for a time of between about 10 minutes to about 15 minutes;
h) storing the frozen sperm at a temperature of between about −180° to about −220° C. for a desired time period; and
i) thawing said sperm, to thereby provide sperm.

25. The method of claim 24, wherein the sperm are thawed for about 90 seconds in a water bath at about 37° C.

26. A method of making an animal, comprising fertilizing an oocyte with sperm preserved by the method of claim 1, claim 13, or claim 24.
